(19) United States
(12) Patent Application Publication
(10) Pub. No.: US 2024/0239202 A1
(43) Pub. Date: Jul. 18, 2024

(54) DEVICE AND METHOD FOR DIAGNOSING HYDROGEN LEAKAGE OF FUEL CELL VEHICLE

(71) Applicants: HYUNDAI MOTOR COMPANY, Seoul (KR); KIA CORPORATION, Seoul (KR)

(72) Inventors: Chang Hwa Hyun, Seoul (KR); Hyun Hie Kim, Seoul (KR); Jong Gyun Kim, Yongin-si (KR)

(21) Appl. No.: 18/207,915

(22) Filed: Jun. 9, 2023

(30) Foreign Application Priority Data
Jan. 16, 2023 (KR) 10-2023-0006227

(51) Int. Cl. B60L 3/00; B60L 58/32; H01M 8/0444; H01M 8/04664

(52) U.S. Cl. CPC B60L 3/0053; B60L 58/32; H01M 8/0444; H01M 8/04679; H01M 2250/20

(57) ABSTRACT

A device for diagnosing hydrogen leakage of a fuel cell vehicle includes a hydrogen sensor for measuring a concentration of hydrogen, and a controller that detects the hydrogen leakage via the hydrogen sensor, controls an air supercharger for a predetermined duration, and diagnoses a cause of the hydrogen leakage based on a ratio of a hydrogen concentration before air supercharging to a hydrogen concentration after the air supercharging.

### CROSS REFERENCE TO RELATED APPLICATIONS

This application claims the benefit of priority to Korean Patent Application No. 10-2023-0006227, filed in the Korean Intellectual Property Office on Jan. 16, 2023, the entire contents of which are incorporated herein by reference.

### TECHNICAL FIELD

The present disclosure relates to a technology for diagnosing a cause of hydrogen leakage of a fuel cell vehicle.

### BACKGROUND

In general, a fuel cell vehicle is equipped with a fuel cell system including a fuel cell stack in which a plurality of fuel cells are stacked, a fuel supply system for supplying hydrogen or the like, which is a fuel, to the fuel cell stack, an air supply system for supplying oxygen, which is an oxidizing agent, required for an electrochemical reaction, a water/thermal management system for controlling a temperature of the fuel cell stack, a battery management system for storing energy produced by the fuel cell stack, and the like.

In this regard, the fuel supply system depressurizes compressed hydrogen inside a hydrogen tank and supplies the hydrogen to an anode of the stack, and the air supply system operates an air blower to supply sucked external air to a cathode of the stack.

When the hydrogen is supplied to the anode of the fuel cell stack and oxygen is supplied to the cathode of the fuel cell stack, hydrogen ions are separated via a catalytic reaction. The separated hydrogen ions are transferred to an oxidation electrode, which is the cathode, via an electrolyte membrane, and the hydrogen ions separated from the anode, electrons, and the oxygen cause an electrochemical reaction together at the oxidation electrode, so that electrical energy may be obtained. Specifically, electrochemical oxidation of the hydrogen occurs at the anode, and electrochemical reduction of the oxygen occurs at the cathode. Electricity and heat are generated by a migration of the electrons generated at this time, and steam or water is produced by a chemical action of combination of the hydrogen and the oxygen.

A discharge apparatus is disposed to discharge byproducts, such as water vapor, the water, and the heat generated during the electrical energy generation process of the fuel cell stack, and the hydrogen, the oxygen, and the like that are not reacted, and gases such as the water vapor, the hydrogen, and the oxygen are discharged into atmosphere via an exhaust passage.

Components, such as the air blower, a hydrogen recirculation blower, and a water pump, for driving the fuel cell may be connected to a main bus bar to facilitate starting the fuel cell. Various relays for facilitating power cut-off and connection and diodes for preventing reverse current from flowing into the fuel cell may be connected to the main bus bar.

The dry air supplied via the air blower may be humidified via an air humidifier and then supplied to the cathode of the fuel cell stack, and the exhaust gas of the cathode may be delivered to the air humidifier in a state of being humidified by the water component generated inside and may be used to humidify the dry air to be supplied to the cathode by the air blower.

To minimize a risk of explosion or ignition caused by hydrogen leakage, such a fuel cell vehicle is equipped with a hydrogen sensor (or a hydrogen detection sensor) on a fuel tank, the fuel cell stack, a hydrogen supply line, and the like, and warns of the hydrogen leakage when a hydrogen concentration detected by the hydrogen sensor exceeds a reference value.

As a result, the conventional fuel cell vehicle can simply warn only of the hydrogen leakage, but cannot diagnose a cause of the hydrogen leakage, and cannot diagnose whether an offset of the hydrogen sensor has occurred.

The matters described in this background art section are written to promote understanding of the background of the invention, and are able to include matters that are not the prior art already known to those skilled in the art in the field to which this technology belongs.

### SUMMARY

The present disclosure has been made to solve the above-mentioned problems occurring in the prior art while advantages achieved by the prior art are maintained intact.

An aspect of the present disclosure provides a device and a method for diagnosing hydrogen leakage of a fuel cell vehicle that may diagnose a cause of the hydrogen leakage with high accuracy by detecting the hydrogen leakage via a hydrogen sensor, controlling an air supercharger for a predetermined duration, and diagnosing the cause of the hydrogen leakage based on a ratio of a hydrogen concentration after air supercharging to a hydrogen concentration before the air supercharging.

Another aspect of the present disclosure is to operate an air compressor or a radiator fan to supercharge air, and diagnose a hydrogen backflow at an air humidifier, micro leakage of a fuel supply valve (FSV), and an occurrence of an offset of a hydrogen sensor based on a ratio of a hydrogen concentration after the air supercharging to a hydrogen concentration before the air supercharging.

Another aspect of the present disclosure is to operate an air compressor to supercharge air when a hydrogen sensor disposed in a fuel cell stack detects hydrogen leakage, and diagnose a hydrogen backflow at an air humidifier, micro leakage of a fuel supply valve (FSV), and an occurrence of an offset of a hydrogen sensor with high accuracy based on a ratio of a hydrogen concentration after the air supercharging to a hydrogen concentration before the air supercharging.

Another aspect of the present disclosure is to operate a radiator fan to supercharge air when a hydrogen sensor disposed in a hydrogen supply line detects hydrogen leakage, and diagnose a hydrogen backflow at an air humidifier, micro leakage of a fuel supply valve (FSV), and an occurrence of an offset of a hydrogen sensor with high accuracy based on a ratio of a hydrogen concentration after the air supercharging to a hydrogen concentration before the air supercharging.

Another aspect of the present disclosure is to improve maintenance efficiency of a fuel cell vehicle by turning on a warning light indicating an abnormality in a hydrogen supply line when hydrogen leakage occurs by the micro leakage of an FSV.

Another aspect of the present disclosure is to improve maintenance efficiency of a fuel cell vehicle by turning on a warning light indicating an abnormality in a hydrogen sensor when an offset occurs in the hydrogen sensor.

The technical problems to be solved by the present disclosure are not limited to the aforementioned problems, and any other technical problems not mentioned herein will be clearly understood from the following description by those skilled in the art to which the present disclosure pertains.

According to an aspect of the present disclosure, a device for diagnosing hydrogen leakage of a fuel cell vehicle includes a hydrogen sensor for measuring a concentration of hydrogen, and a controller that detects the hydrogen leakage via the hydrogen sensor, controls an air supercharger for a predetermined duration, and diagnoses a cause of the hydrogen leakage based on a ratio of a hydrogen concentration before air supercharging to a hydrogen concentration after the air supercharging.

In one implementation, the controller may determine the cause of the hydrogen leakage as a hydrogen backflow at an air humidifier when the ratio satisfies a first condition, determine that an offset has occurred in the hydrogen sensor when the ratio satisfies a second condition, and determine the cause of the hydrogen leakage as micro leakage of a fuel supply valve (FSV) when the ratio satisfies a third condition.

In one implementation, the controller may turn on a warning light indicating an abnormality in a hydrogen supply line when the hydrogen leakage occurs by the micro leakage of the FSV.

In one implementation, the controller may turn on a warning light indicating an abnormality in the hydrogen sensor when the offset occurs in the hydrogen sensor.

In one implementation, the controller may repeatedly perform a process of maintaining an ON state of the air supercharger for a first duration and then maintaining an OFF state of the air supercharger for a second duration during the predetermined duration.

In one implementation, the air supercharger may include at least one of an air compressor and a radiator fan, and the hydrogen sensor may include at least one of a first hydrogen sensor disposed in a fuel cell stack and a second hydrogen sensor disposed in a hydrogen supply line.

In one implementation, the controller may operate the air compressor when the hydrogen leakage is detected via the first hydrogen sensor.

In one implementation, the controller may operate the radiator fan when the hydrogen leakage is detected via the second hydrogen sensor.

In one implementation, the controller may start the diagnosis process when a temperature of a fuel cell stack exceeds a reference temperature.

In one implementation, the controller may detect the hydrogen leakage when the hydrogen concentration measured by the hydrogen sensor exceeds a reference concentration.

According to another aspect of the present disclosure, a method for diagnosing hydrogen leakage of a fuel cell vehicle includes measuring, by a hydrogen sensor, a concentration of hydrogen, detecting, by a controller, the hydrogen leakage via the hydrogen sensor, controlling, by the controller, an air supercharger for a predetermined duration, and diagnosing, by the controller, a cause of the hydrogen leakage based on a ratio of a hydrogen concentration before air supercharging to a hydrogen concentration after the air supercharging.

In one implementation, the diagnosing of the cause of the hydrogen leakage may include determining, by the controller, the cause of the hydrogen leakage as a hydrogen backflow at an air humidifier when the ratio satisfies a first condition, determining, by the controller, that an offset has occurred in the hydrogen sensor when the ratio satisfies a second condition, and determining, by the controller, the cause of the hydrogen leakage as micro leakage of a fuel supply valve (FSV) when the ratio satisfies a third condition.

In one implementation, the diagnosing of the cause of the hydrogen leakage may further include turning, by the controller, on a warning light indicating an abnormality in a hydrogen supply line when the hydrogen leakage occurs by the micro leakage of the FSV.

In one implementation, the diagnosing of the cause of the hydrogen leakage may further include turning, by the controller, on a warning light indicating an abnormality in the hydrogen sensor when the offset occurs in the hydrogen sensor.

In one implementation, the controlling of the air supercharger may include a first maintaining operation of maintaining, by the controller, an ON state of the air supercharger for a first duration, a second maintaining operation of maintaining, by the controller, an OFF state of the air supercharger for a second duration, and repeatedly performing, by the controller, the first maintaining operation and the second maintaining operation during the predetermined duration.

In one implementation, the air supercharger may include at least one of an air compressor and a radiator fan, and the hydrogen sensor may include at least one of a first hydrogen sensor disposed in a fuel cell stack and a second hydrogen sensor disposed in a hydrogen supply line.

In one implementation, the controlling of the air supercharger may include operating, by the controller, the air compressor when the hydrogen leakage is detected via the first hydrogen sensor.

In one implementation, the controlling of the air supercharger may include operating, by the controller, the radiator fan when the hydrogen leakage is detected via the second hydrogen sensor.

In one implementation, the diagnosing of the cause of the hydrogen leakage may include starting, by the controller, the diagnosis of the hydrogen leakage when a temperature of a fuel cell stack exceeds a reference temperature.

In one implementation, the detecting of the hydrogen leakage may include detecting, by the controller, the hydrogen leakage when the hydrogen concentration measured by the hydrogen sensor exceeds a reference concentration.

### BRIEF DESCRIPTION OF THE FIGURES

The above and other objects, features and advantages of the present disclosure will be more apparent from the following detailed description taken in conjunction with the accompanying drawings:

FIG. 1 is an exemplary view of a fuel cell system equipped in a fuel cell vehicle to which an embodiment of the present disclosure is applied;

FIG. 2 is a block diagram of a hydrogen leakage diagnosing device of a fuel cell vehicle according to an embodiment of the present disclosure;

FIG. 3 is a flowchart of a hydrogen leakage diagnosing method of a fuel cell vehicle according to an embodiment of the present disclosure;

FIG. 4 is an exemplary view showing a change in a hydrogen concentration based on operation of an air supercharger in a state in which hydrogen discharged to an air humidifier flows backward;

FIG. 5 is an exemplary diagram showing a change in a hydrogen concentration based on operation of an air supercharger in a state in which an offset has occurred in a hydrogen sensor; and

FIG. 6 is a block diagram showing a computing system for executing a hydrogen leakage diagnosing method of a fuel cell vehicle according to an embodiment of the present disclosure.

### DETAILED DESCRIPTION

Hereinafter, some embodiments of the present disclosure will be described in detail with reference to the exemplary drawings. In adding the reference numerals to the components of each drawing, it should be noted that the identical or equivalent component is designated by the identical numeral even when they are displayed on other drawings. Further, in describing the embodiment of the present disclosure, a detailed description of the related known configuration or function will be omitted when it is determined that it interferes with the understanding of the embodiment of the present disclosure.

In describing the components of the embodiment according to the present disclosure, terms such as first, second, A, B, (a), (b), and the like may be used. These terms are merely intended to distinguish the components from other components, and the terms do not limit the nature, order or sequence of the components. Unless otherwise defined, all terms including technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

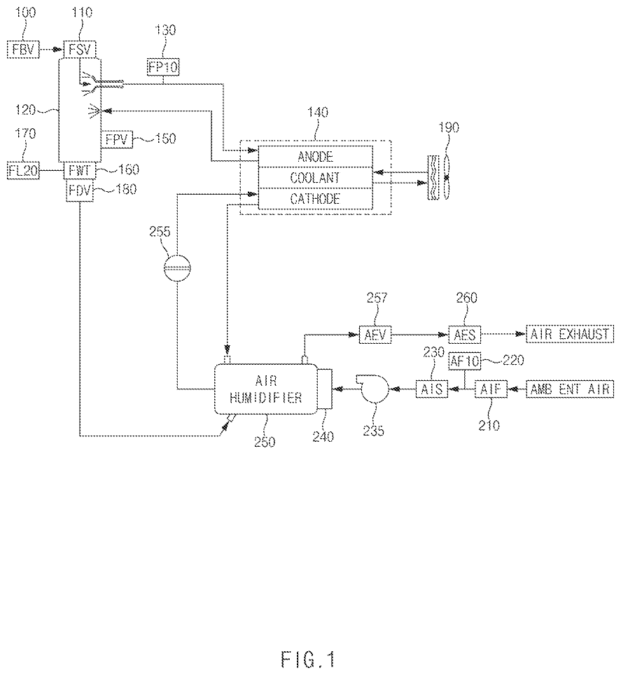

FIG. 1 is an exemplary view of a fuel cell system equipped in a fuel cell vehicle to which an embodiment of the present disclosure is applied. The fuel cell vehicle may include a hydrogen-electric hybrid vehicle (e.g., a truck, a bus, a high-performance vehicle, a tram, and the like).

As shown in FIG. 1, the fuel cell system equipped in the fuel cell vehicle to which the embodiment of the present disclosure is applied may include a fuel block valve (FBV) 100, a fuel supply valve (FSV) 110, a fuel ejector (FEJ) 120, a fuel pressure sensor 10 (FP10) 130, a fuel cell stack 140, a fuel-line purge valve (FPV) 150, a fuel-line water trap (FWT) 160, a fuel-line level sensor (FL20) 170, a fuel-line drain valve (FDV) 180, a radiator fan 190, an air intake filter (AIF) 210, an air flow sensor (AF10) 220, an air intake silencer (AIS) 230, an air compressor 235, an air cooler 240, an air humidifier 250, an air cut-off valve (ACV) 255, an air exhaust valve (AEV) 257, and an air exhaust silencer (AES) 260.

In this regard, the FBV 100 serves to block hydrogen supplied to the fuel cell stack 140. The FSV 110 serves to adjust a pressure of the hydrogen supplied to the fuel cell stack 140. The FEJ 120 serves to apply the pressure to the hydrogen and supply the hydrogen to the fuel cell stack 140. The FP10 130 serves to measure the pressure of the hydrogen supplied to the fuel cell stack 140. The FPV 150 serves to discharge hydrogen electrode condensed water and impurities in the fuel cell stack 140. The FWT 160 serves to store water. The FL20 170 serves to measure a level of the water stored in the FWT 160. The FDV 180 serves to discharge the water stored in the FWT 160.

In addition, the AIF 210 serves to filter out foreign substances (dust or the like) contained in ambient air. The AF10 220 is a sensor that measures a flow rate of air. The AIS 230 removes noise from an intake. The air compressor 235 serves to supply the ambient air to the air humidifier 250. The air cooler 240 serves to cool the air supplied to the air humidifier 250. The air humidifier 250 serves to adjust humidity of the air. The ACV 255 serves to block air supplied to a cathode of the fuel cell stack 140. The AEV 257 serves to discharge the hydrogen from the cathode to the outside via an air exhaust line. The AES 260 eliminates noise generated when exhaust gas is discharged via the air exhaust line.

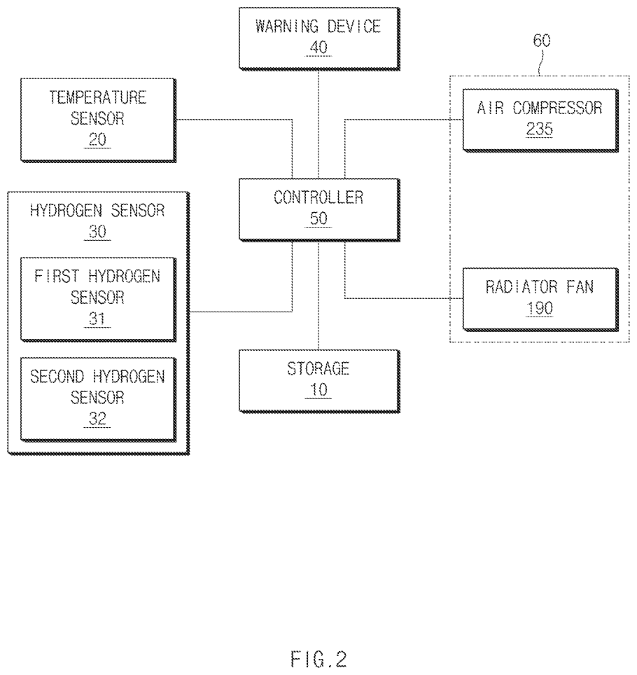

FIG. 2 is a block diagram of a hydrogen leakage diagnosing device of a fuel cell vehicle according to an embodiment of the present disclosure.

As shown in FIG. 2, the hydrogen leakage diagnosing device of the fuel cell vehicle according to one embodiment of the present disclosure may include storage 10, a temperature sensor 20, a hydrogen sensor 30, a warning device 40, a controller 50, and an air supercharger 60. In this regard, the components may be combined with each other and implemented as one component and some components may be omitted based on a scheme for implementing the hydrogen leakage diagnosing device of the fuel cell vehicle according to one embodiment of the present disclosure. In addition, the air compressor 235 and the radiator fan 190 are referred to as the air supercharger 60.

Each of the above components will be described. First, the storage 10 may store various logics, algorithms, and programs required in a process of detecting hydrogen leakage via the hydrogen sensor 30, controlling the air supercharger 60 for a predetermined duration, and diagnosing a cause of the hydrogen leakage based on a ratio of a hydrogen concentration after air supercharging to a hydrogen concentration before the air supercharging.

The storage 10 may store various logics, algorithms, and programs required in a process of supercharging the air by operating the air compressor 235 or the radiator fan 190 and diagnosing a hydrogen backflow at the air humidifier 250, micro leakage of the FSV 110, and an occurrence of an offset of the hydrogen sensor based on the ratio of the hydrogen concentration after the air supercharging to the hydrogen concentration before the air supercharging.

The storage 10 may store various logics, algorithms, and programs required in a process of operating the air compressor 235 to supercharge the air when a first hydrogen sensor 31 disposed in the fuel cell stack 140 detects the hydrogen leakage and operating the radiator fan 190 to supercharge the air when a second hydrogen sensor 32 disposed in a hydrogen supply line detects the hydrogen leakage.

The storage 10 may store various logics, algorithms, and programs required in a process of controlling the warning device 40 to turn on a warning light indicating an abnormality in the hydrogen supply line when the hydrogen leakage occurs by the micro leakage of the FSV 110 and controlling the warning device 40 to turn on a warning light indicating an abnormality in the hydrogen sensor 30 when the offset occurs in the hydrogen sensor.

The storage 10 may include at least one of recording media (storage media) such as a memory of a flash memory type, a hard disk type, a micro type, a card type (e.g., a secure digital card (SD card) or an extreme digital card (XD card)), and the like, and a memory of a random access memory (RAM) type, a static RAM (SRAM) type, a read-only memory (ROM) type, a programmable ROM (PROM) type, an electrically erasable PROM (EEPROM) type, a magnetic RAM (MRAM) type, a magnetic disk type, and an optical disk type.

The temperature sensor 20 may be disposed in the fuel cell stack 140 to measure a temperature of the fuel cell stack 140.

The hydrogen sensor 30 may measure a concentration of the hydrogen leaked from the fuel cell vehicle. Such hydrogen sensor 30 may include the first hydrogen sensor 31 disposed in the fuel cell stack 140 and the second hydrogen sensor 32 disposed in the hydrogen supply line.

The warning device 40 may include the warning light indicating the abnormality in the hydrogen supply line and the warning light indicating the abnormality in the hydrogen sensor 30.

The controller 50 may perform overall control such that each of the components may normally perform a function thereof. Such controller 50 may be implemented in a form of hardware, software, or a combination of the hardware and the software. Preferably, the controller 50 may be implemented as a microprocessor, but the present disclosure may not be limited thereto.

In particular, the controller 50 may detect the hydrogen leakage via the hydrogen sensor 30, control the air supercharger 60 for the predetermined duration, and diagnose the cause of the hydrogen leakage based on the ratio of the hydrogen concentration after the air supercharging to the hydrogen concentration before the air supercharging. In this regard, the controller 50 may start a process of diagnosing the cause of the hydrogen leakage when the temperature of the fuel cell stack 140 exceeds a reference temperature (that is, when the fuel cell stack is warmed up).

The controller 50 may repeatedly perform a process of maintaining an ON state of the air supercharger 60 for a first duration (e.g., 30 seconds) and then maintaining an OFF state of the air supercharger 60 for a second duration (e.g., 10 seconds) for the predetermined duration. In this regard, the controller 50 may maintain an RPM of the air supercharger 60 at, for example, 50,000 in the ON state of the air supercharger 60.

The controller 50 may operate the air compressor 235 or the radiator fan 190 to supercharge the air, and diagnose the hydrogen backflow at the air humidifier 250, the micro leakage of the FSV 110, and the occurrence of the offset of the hydrogen sensor based on the ratio of the hydrogen concentration after the air supercharging to the hydrogen concentration before the air supercharging.

The controller 50 may operate the air compressor 235 to supercharge the air when the first hydrogen sensor 31 disposed in the fuel cell stack 140 detects the hydrogen leakage, and operate the radiator fan 190 to supercharge the air when the second hydrogen sensor 32 disposed in the hydrogen supply line detects the hydrogen leakage.

The controller 50 may control the warning device 40 to turn on the warning light indicating the abnormality in the hydrogen supply line when the hydrogen leakage occurs by the micro leakage of the FSV 110, and control the warning device 40 to turn on the warning light indicating the abnormality in the hydrogen sensor 30 when the offset occurs in the hydrogen sensor.

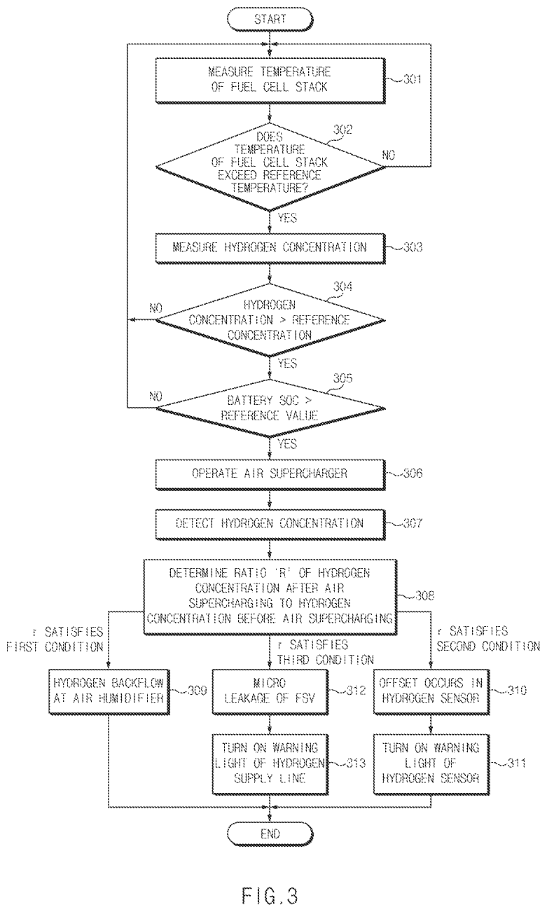

Hereinafter, a diagnosis operation of the controller 50 will be described in detail with reference to FIGS. 3 to 5.

FIG. 3 is a flowchart of a hydrogen leakage diagnosing method of a fuel cell vehicle according to an embodiment of the present disclosure.

First, the temperature sensor 20 may measure the temperature of the fuel cell stack 140 (301).

Thereafter, it is preferable that the controller 50 performs a diagnosis process below when the temperature of the fuel cell stack 140 measured by the temperature sensor 20 exceeds the reference temperature (a predetermined temperature), that is, when the fuel cell stack 140 is in the warmed-up state (302).

Thereafter, the hydrogen sensor 30 may measure the hydrogen concentration (303).

Thereafter, when the hydrogen concentration measured by the hydrogen sensor 30 exceeds a reference concentration (a predetermined concentration) (304), and when a state of charge (SOC) of a battery exceeds a reference value (a predetermined value) (305), the controller 50 may operate the air supercharger 60 for the predetermined duration (306). In this regard, the reason why the controller 50 checks the SOC of the battery is to secure power necessary for operating the air supercharger 60. In this regard, the controller 50 may repeatedly perform the process of maintaining the ON state of the air supercharger 60 for the first duration (e.g., 30 seconds) and then maintaining the OFF state of the air supercharger 60 for the second duration (e.g., 10 seconds) for the predetermined duration.

Thereafter, the hydrogen sensor 30 may measure the hydrogen concentration (307).

Thereafter, the controller 50 may determine a ratio ‘r’ of the hydrogen concentration after the air supercharging to the hydrogen concentration before the air supercharging (308).

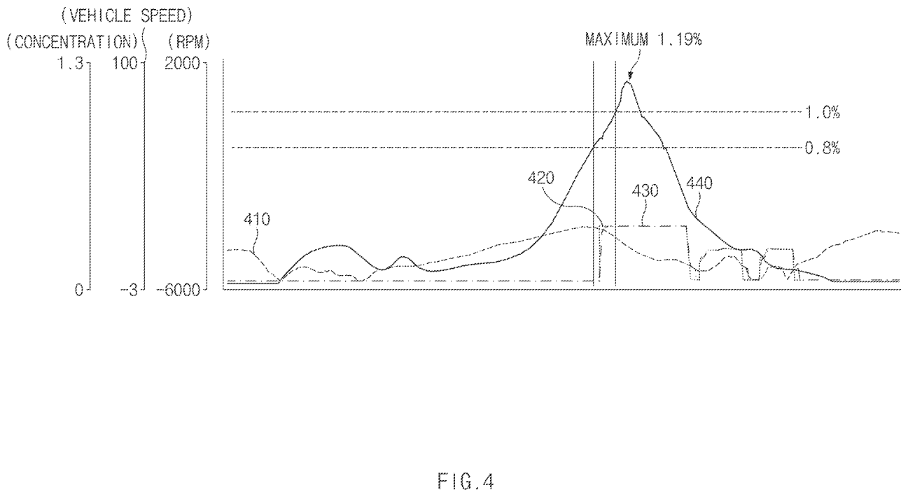

Thereafter, the controller 50 may determine the cause of the hydrogen leakage as the hydrogen backflow at the air humidifier 250 when the ratio ‘r’ satisfies a first condition (e.g., 0<r<0.1) (309). The basis therefor is as shown in FIG. 4.

FIG. 4 is an exemplary view showing a change in a hydrogen concentration based on operation of an air supercharger in a state in which hydrogen discharged to an air humidifier flows backward.

In FIG. 4, a horizontal axis represents time, a vertical axis represents the hydrogen concentration, a vehicle speed, and the revolutions per minute (RPM) of the air supercharger 60, ‘410’ represents the vehicle speed over time, ‘420’ represents an RPM command of the air supercharger 60 over time, ‘430’ represents the actual RPM of the air supercharger 60 over time, and ‘440’ represents the hydrogen concentration.

As shown in FIG. 4, it may be seen that, in a process in which a hydrogen concentration 440 increases as the hydrogen discharged to the air humidifier 250 via a purge and the FDV 180 flows back, the hydrogen concentration 440 gradually converges to 0% as the air supercharger 60 is operated. Eventually, because of the operation of the air supercharger 60, the ratio ‘r’ satisfies the first condition.

In addition, a crossover phenomenon of the hydrogen resulted from accumulation of idle operation without driving the air compressor 235, a discharge of the hydrogen to the air humidifier 250 based on a regular correction of the FP10 130, and an increase in the hydrogen concentration of the cathode and the air humidifier caused by cold shutdown (CSD) in winter and additional drain may cause the hydrogen discharged to the air humidifier 250 to flow backward. In this regard, the crossover phenomenon refers to a phenomenon in which hydrogen gas of an anode is diffused to a cathode because of a gas concentration difference between the anode and the cathode in the fuel cell stack 140, and the CSD refers to a function to improve the ability to start up at low-temperatures in the winter.

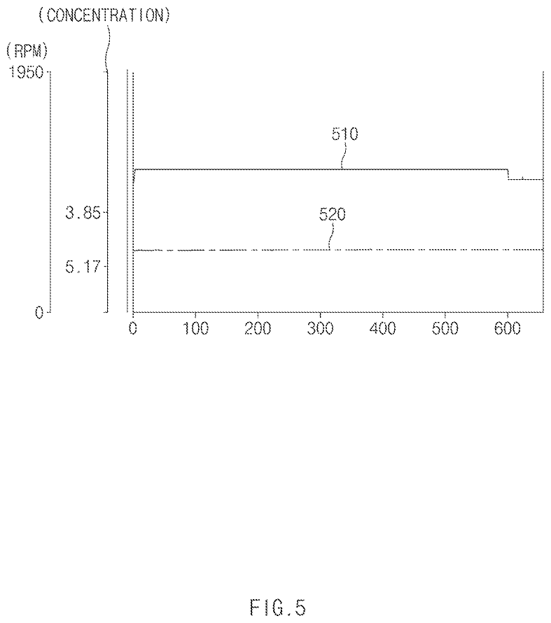

On the other hand, the controller 50 may determine that the offset has occurred in the hydrogen sensor 30 when the ratio ‘r’ satisfies a second condition (e.g., r=1) (310). The basis therefor is as shown in FIG. 5.

FIG. 5 is an exemplary diagram showing a change in a hydrogen concentration based on operation of an air supercharger in a state in which an offset has occurred in a hydrogen sensor.

In FIG. 5, a horizontal axis represents time, a vertical axis represents the hydrogen concentration and the RPM of the air supercharger 60, ‘510’ represents the RPM of the air supercharger 60 over time, and ‘520’ represents the hydrogen concentration over time.

As shown in FIG. 5, it may be seen that, in the state in which the offset (e.g., 0.5%) has occurred in the hydrogen sensor 30, the hydrogen concentration maintains 0.5% despite the air supercharger 60 being operated. In this case, because the hydrogen sensor 30 malfunctions and outputs 0.5% even when the actual hydrogen concentration is 0%, the ratio ‘r’ eventually satisfies the second condition.

Thereafter, the controller 50 may control the warning device 40 to turn on the warning light indicating the abnormality in the hydrogen sensor 30 (311).

On the other hand, when the ratio ‘r’ satisfies a third condition (r≤0 or r≥0.1 and r≠1), the controller 50 may determine the cause of the hydrogen leakage as the micro leakage of the FSV 110 (312). In this regard, the controller 50 may control the warning device 40 to turn on the warning light indicating the abnormality in the hydrogen supply line (313).

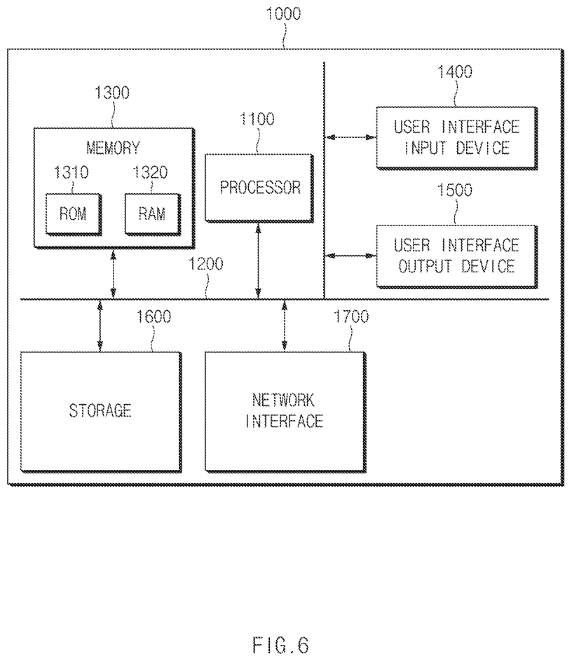

FIG. 6 is a block diagram showing a computing system for executing a hydrogen leakage diagnosing method of a fuel cell vehicle according to an embodiment of the present disclosure.

Referring to FIG. 6, the hydrogen leakage diagnosing method of the fuel cell vehicle according to one embodiment of the present disclosure described above may also be implemented via a computing system. A computing system 1000 may include at least one processor 1100, a memory 1300, a user interface input device 1400, a user interface output device 1500, storage 1600, and a network interface 1700 connected via a system bus 1200.

The processor 1100 may be a central processing unit (CPU) or a semiconductor device that performs processing on commands stored in the memory 1300 and/or the storage 1600. The memory 1300 and the storage 1600 may include various types of volatile or non-volatile storage media. For example, the memory 1300 may include a ROM (Read Only Memory) 1310 and a RAM (Random Access Memory) 1320.

Thus, the operations of the method or the algorithm described in connection with the embodiments disclosed herein may be embodied directly in hardware or a software module executed by the processor 1100, or in a combination thereof. The software module may reside on a storage medium (that is, the memory 1300 and/or the storage 1600) such as a RAM, a flash memory, a ROM, an EPROM, an EEPROM, a register, a hard disk, a removable disk, and a CD-ROM. The exemplary storage medium is coupled to the processor 1100, which may read information from, and write information to, the storage medium. In another method, the storage medium may be integral with the processor 1100. The processor 1100 and the storage medium may reside within an application specific integrated circuit (ASIC). The ASIC may reside within the user terminal. In another method, the processor 1100 and the storage medium may reside as individual components in the user terminal.

The description above is merely illustrative of the technical idea of the present disclosure, and various modifications and changes may be made by those skilled in the art without departing from the essential characteristics of the present disclosure. Therefore, the embodiments disclosed in the present disclosure are not intended to limit the technical idea of the present disclosure but to illustrate the present disclosure, and the scope of the technical idea of the present disclosure is not limited by the embodiments. The scope of the present disclosure should be construed as being covered by the scope of the appended claims, and all technical ideas falling within the scope of the claims should be construed as being included in the scope of the present disclosure.

The device and the method for diagnosing the hydrogen leakage of the fuel cell vehicle according to one embodiment of the present disclosure as described above may diagnose the cause of the hydrogen leakage with the high accuracy by detecting the hydrogen leakage via the hydrogen sensor, controlling the air supercharger for the predetermined duration, and diagnosing the cause of the hydrogen leakage based on the ratio of the hydrogen concentration after the air supercharging to the hydrogen concentration before the air supercharging.

The present disclosure may operate the air compressor or the radiator fan as the air supercharger to supercharge the air, and diagnose the hydrogen backflow at the air humidifier, the micro leakage of the fuel supply valve (FSV), and the occurrence of the offset of the hydrogen sensor based on the ratio of the hydrogen concentration after the air supercharging to the hydrogen concentration before the air supercharging.

The present disclosure may improve the maintenance efficiency of the fuel cell vehicle by turning on the warning light indicating the abnormality in the hydrogen supply line when the hydrogen leakage occurs because of the micro leakage of the FSV and turning on the warning light indicating the abnormality in the hydrogen sensor when the offset occurs in the hydrogen sensor.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

1. A device for diagnosing hydrogen leakage of a fuel cell vehicle, the device comprising:
a hydrogen sensor configured to measure a concentration of hydrogen; and
a controller configured to:
detect hydrogen leakage via the hydrogen sensor;
control an air supercharger for a predetermined duration; and
diagnose a cause of the hydrogen leakage based on a ratio of a hydrogen concentration before air supercharging to a hydrogen concentration after the air supercharging.

2. The device of claim 1, wherein the controller is further configured to:
determine the cause of the hydrogen leakage as a hydrogen backflow at an air humidifier when the ratio satisfies a first condition;
determine that an offset has occurred in the hydrogen sensor when the ratio satisfies a second condition; and
determine the cause of the hydrogen leakage as micro leakage of a fuel supply valve (FSV) when the ratio satisfies a third condition.

3. The device of claim 2, wherein the controller is further configured to turn on a warning light indicating an abnormality in a hydrogen supply line when the hydrogen leakage occurs by the micro leakage of the FSV.

4. The device of claim 2, wherein the controller is further configured to turn on a warning light indicating an abnormality in the hydrogen sensor when the offset occurs in the hydrogen sensor.

5. The device of claim 1, wherein the controller is further configured to repeatedly perform a process of maintaining an ON state of the air supercharger for a first duration, and then maintaining an OFF state of the air supercharger for a second duration during the predetermined duration.

6. The device of claim 1, wherein the air supercharger includes at least one of an air compressor and a radiator fan, and the hydrogen sensor includes at least one of a first hydrogen sensor disposed in a fuel cell stack and a second hydrogen sensor disposed in a hydrogen supply line.

7. The device of claim 6, wherein the controller is further configured to operate the air compressor when the hydrogen leakage is detected via the first hydrogen sensor.

8. The device of claim 6, wherein the controller is further configured to operate the radiator fan when the hydrogen leakage is detected via the second hydrogen sensor.

9. The device of claim 1, wherein the controller is further configured to start the diagnosing when a temperature of a fuel cell stack exceeds a reference temperature.

10. The device of claim 1, wherein the controller is further configured to detect the hydrogen leakage when the hydrogen concentration measured by the hydrogen sensor exceeds a reference concentration.

11. A method for diagnosing hydrogen leakage of a fuel cell vehicle, the method comprising:
measuring, by a hydrogen sensor, a concentration of hydrogen;
detecting, by a controller, hydrogen leakage via the hydrogen sensor;
controlling, by the controller, an air supercharger for a predetermined duration; and
diagnosing, by the controller, a cause of the hydrogen leakage based on a ratio of a hydrogen concentration before air supercharging to a hydrogen concentration after the air supercharging.

12. The method of claim 11, wherein diagnosing the cause of the hydrogen leakage includes:
determining, by the controller, the cause of the hydrogen leakage as a hydrogen backflow at an air humidifier when the ratio satisfies a first condition;
determining, by the controller, that an offset has occurred in the hydrogen sensor when the ratio satisfies a second condition; and
determining, by the controller, the cause of the hydrogen leakage as micro leakage of a fuel supply valve (FSV) when the ratio satisfies a third condition.

13. The method of claim 12, wherein diagnosing the cause of the hydrogen leakage further includes:
turning on, by the controller, a warning light indicating an abnormality in a hydrogen supply line when the hydrogen leakage occurs by the micro leakage of the FSV.

14. The method of claim 12, wherein diagnosing the cause of the hydrogen leakage further includes:
turning on, by the controller, a warning light indicating an abnormality in the hydrogen sensor when the offset occurs in the hydrogen sensor.

15. The method of claim 11, wherein controlling the air supercharger includes:
a first maintaining operation of maintaining, by the controller, an ON state of the air supercharger for a first duration;
a second maintaining operation of maintaining, by the controller, an OFF state of the air supercharger for a second duration; and
repeatedly performing, by the controller, the first maintaining operation and the second maintaining operation during the predetermined duration.

16. The method of claim 11, wherein the air supercharger includes at least one of an air compressor and a radiator fan, and the hydrogen sensor includes at least one of a first hydrogen sensor disposed in a fuel cell stack and a second hydrogen sensor disposed in a hydrogen supply line.

17. The method of claim 16, wherein controlling the air supercharger includes:
operating, by the controller, the air compressor when the hydrogen leakage is detected via the first hydrogen sensor.

18. The method of claim 16, wherein controlling the air supercharger includes:
operating, by the controller, the radiator fan when the hydrogen leakage is detected via the second hydrogen sensor.

19. The method of claim 11, wherein diagnosing the cause of the hydrogen leakage includes:
starting, by the controller, the diagnosing of the hydrogen leakage when a temperature of a fuel cell stack exceeds a reference temperature.

20. The method of claim 11, wherein detecting the hydrogen leakage includes:
detecting, by the controller, the hydrogen leakage when the hydrogen concentration measured by the hydrogen sensor exceeds a reference concentration.
